(19) United States
(12) Patent Application Publication
(10) Pub. No.: US 2005/0191683 A1
(43) Pub. Date: Sep. 1, 2005

(54) Biomolecule sensing method using Field Effect Transistor with controllable Debye length

(76) Inventors: Kyu-tae Yoo, Seoul (KR); Geun-bae Lim, Pohang-si (KR)

Correspondence Address: CANTOR COLBURN, LLP, 55 GRIFFIN ROAD SOUTH, BLOOMFIELD, CT, 06002, US

(21) Appl. No.: 11/063,214

(22) Filed: Feb. 22, 2005

(30) Foreign Application Priority Data
Feb. 27, 2004 (KR) 10-2004-0013680

(51) Int. Cl. C12Q 1/68

(52) U.S. Cl. 435/6

(57) ABSTRACT

A method of sensing biomolecules in an electrolyte solution by using a bio FET. When it is sensed that probe biomolecules are immobilized to a gate surface of the bio FET or that the probe biomolecules are hybridized with target biomolecules, a Debye length from the biomolecules having charges attached to the gate surface is controlled.

### BACKGROUND OF THE INVENTION

This application claims the priority of Korean Patent Application No. 10-2004-0013680, filed on Feb. 27, 2004, in the Korean Intellectual Property Office, the disclosure of which is incorporated herein in its entirety by reference.

1. Field of the Invention

The present invention relates to a method of sensing whether or not biomolecules are immobilized using a bio Field Effect Transistor (FET), and more particularly, to a method of electrically sensing whether or not probe biomolecules are attached to a gate surface of a bio FET, and whether or not the probe biomolecules are bonded with target biomolecules.

2. Description of the Related Art

Among sensors for sensing biomolecules by an electric signal, there is a transistor-based biosensor having a structure with a transistor (TR). The biosensor manufactured using a semiconductor process has an advantage of fast electric signal conversion and of easy connection of an Integrated Circuit (IC) and a MicroElectroMechanical System (MEMS). Accordingly, many studies on the biosensor are being made up to now.

U.S. Pat. No. 4,238,757 filed in 1980 is an original patent disclosing a sensing of a biological reaction using a FET. The U.S. Pat. No. 4,238,757 discloses a biosensor for sensing an antigen-antibody reaction by a current variation of a semiconductor inversion layer, and more particularly, discloses a protein among biomolecules. The current variation is caused by a variation of a surface charge strength. U.S. Pat. No. 4,777,019 filed in 1986 discloses that biological monomers are absorbed on a gate surface to sense a degree of hybridization with a complementary monomer by using the FET. U.S. Pat. No. 5,846,708 filed in 1998 discloses a method for sensing whether or not hybridization is performed, using a light absorption phenomenon caused by biomolecules that are bonded using a charged coupled device (CCD). U.S. Pat. Nos. 5,466,348 and 6,203,981 using the TFT disclose a circuit connection to improve a ratio of signal to noise.

In case where the TFT is used, there is an advantage in that a cost is reduced in comparison with a transistor formed in a silicon substrate, and in that a chip can be manufactured to have an array format for increasing a substrate area, thereby improving an integration degree. In case where the FET is used as the biosensor, there is an advantage of less cost and time, and an easy association with an integrated circuit/MEMS process.

FIG. 1 is a sectional view illustrating a conventional typical bio FET. A source region 12a and a drain region 12b are formed at both sides of an n-type or p-type doped substrate 11 to have an opposite type of doping to the substrate 11. A gate 13 is formed to be in contact with the source region 12a and the drain region 12b. Here, the gate 13 is generally formed of an oxidation layer 14, a poly silicon layer 15 and a metal layer 16. Probe biomolecules 17 are attached to the gate electrode layer 16. The probe biomolecules 17 are bonded with predetermined target biomolecules through a hydrogen bond and the like. The bonding is sensed by an electrical method to sense a bonding degree of the probe biomolecules 17 with the target biomolecules.

However, the above U.S. patents have a drawback in that accuracy and reappearance are not reliably assured when the charged biomolecules are sensed in an electrolyte solution 10. In detail, the probe biomolecules 17 are bonded to a gate 13 surface of the bio FET, or target biomolecules are bonded to the probe biomolecules 17 in the electrolyte solution 10. At this time, in case where the charged biomolecules are distant away from the gate 13 surface by a predetermined distance (Debye length) due to an ionic shielding phenomenon occurring between ions of the electrolyte solution 10, an electrical potential of the gate 13 surface is not affected. Accordingly, it is difficult to accurately sense a potential of the gate 13 surface. Accordingly, it is sensed with poor reappearance and accuracy whether or not the probe biomolecules 17 are immobilized to the gate 13 surface and whether or not the probe biomolecules 17 are hybridized with the target biomolecules.

In U.S. Pat. No. 6,355,436, a voltage variation between a gate and a source region due to hybridization is sensed. Additionally, the voltage variation is sensed between the gate and the source region depending on a strength of the target biomolecules. U.S. Pat. No. 6,482,639 B2 discloses a sensing of uncharged biomolecules as well as charged biomolecules. The biomolecules are sensed by a capacitance variation caused by absorption/bond of the biomolecules between a reference electrode and a gate surface. However, the above U.S. patents have a drawback in that the reappearance and the accuracy are poor when the bio FET is sensed.

### SUMMARY OF THE INVENTION

The present invention provides a biomolecule sensing method using a bio FET in which it is accurately sensed whether or not probe biomolecules are immobilized to a surface of the bio FET and whether or not target biomolecules are hybridized with the probe biomolecules.

According to an aspect of the present invention, there is provided a method of sensing biomolecules in an electrolyte solution by using a bio FET, wherein when it is sensed that probe biomolecules are immobilized to a gate surface of the bio FET or that the probe biomolecules are hybridized with target biomolecules, a Debye length from the biomolecules having charges attached to the gate surface is controlled.

The present invention is characterized in that when it is sensed that the probe biomolecules are immobilized to the gate surface of the bio FET, the Debye length is decreased.

The present invention is characterized in that when it is sensed that the immobilized probe biomolecules are hybridized with the target biomolecules, the Debye length is increased.

The present invention is characterized in that the Debye length is varied by controlling an ionic strength of the electrolyte solution.

The present invention is characterized in that the Debye length is varied by controlling a temperature.

According to another aspect of the present invention, there is provided a method of sensing biomolecules in an electrolyte solution by using a bio FET, the method including: when it is sensed that probe biomolecules are immobilized to a gate surface, adding a high ionic strength of solution to the electrolyte solution to reduce a Debye length from the probe biomolecules having charges, and then sensing a current flowing via a channel region between a source region and a drain region of the bio FET; and when it is sensed that target biomolecules are hybridized with the probe bio molecules, adding a low ionic strength of solution to the electrolyte solution to increase the Debye length from the target biomolecules having charges, and then sensing a current flowing via the channel region between the source region and the drain region of the bio FET.

### BRIEF DESCRIPTION OF THE DRAWINGS

The above and other features and advantages of the present invention will become more apparent by describing in detail exemplary embodiments thereof with reference to the attached drawings in which:

FIG. 1 is a sectional view illustrating a conventional typical bio FET;

FIG. 2 is a view illustrating immobilization of probe DNAs on a gate of a bio FET;

FIGS. 3A and 3B are views illustrating a sensing of current flowing via a channel region between a source region and a drain region depending on a variation of Debye length in case where probe biomolecules are immobilized on a gate surface;

FIGS. 4A and 4B are views illustrating a sensing of current flowing via a channel region between a source region and a drain region depending on a variation of Debye length in case where probe biomolecules are hybridized with target biomolecules;

FIG. 5A is a view illustrating a micro channel for supplying or discharging an electrolyte solution with biomolecules to or from a gate surface of a bio FET;

FIG. 5B is a graph illustrating a real-time sensing of current flowing via a channel region between a source region and a drain region depending on a controllable Debye length in case where probe biomolecules are immobilized on a gate surface of a bio FET or are hybridized with target biomolecules.

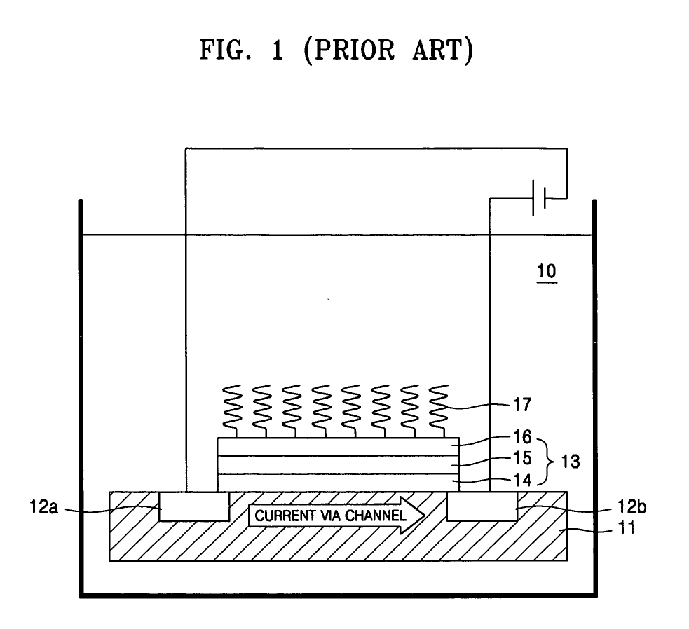

### DETAILED DESCRIPTION OF THE INVENTION

The present invention will now be described more fully with reference to the accompanying drawings, in which exemplary embodiments of the invention are shown.

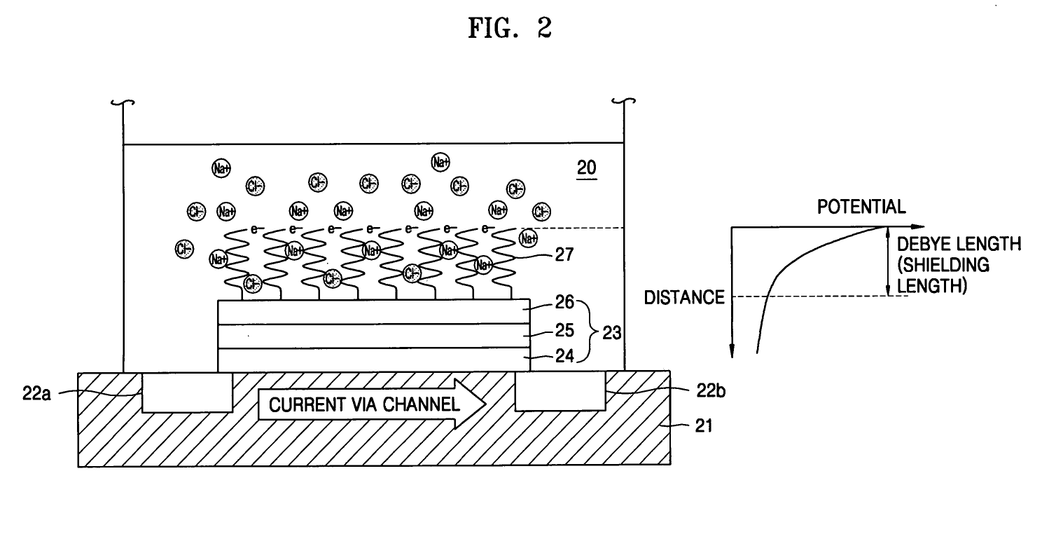

FIG. 2 is a view illustrating immobilization of probe DNAs 27 on a gate 23 of a bio FET installed in an electrolyte solution 20.

Referring to FIG. 2, impurity-doped source region 22a and drain region 22b are formed at both sides of a substrate 21. A gate 23 is formed on the substrate 21 to be in contact with the source region 22a and the drain region 22b. The gate 23 has no limit in shape, but is generally comprised of a gate insulating layer 24, a gate electrode layer 25, and a metal layer 26 for attaching probe biomolecules 27.

The bio FET senses a variation of current flowing via a channel region of the substrate, which is caused by a surface charge density of the probe biomolecules immobilized on a gate 23 surface, such that it is sensed depending on its variation whether or not the probe biomolecules 27 are immobilized and whether or not the probe biomolecules 27 are hybridized with the target biomolecules.

As shown in FIG. 2, the probe biomolecules, for example, the probe DNAs 27 are charged and immobilized on the substrate 21. As the attached probe DNAs 27 is increased in amount, the surface charge density is increased. Accordingly, the current flowing via the channel region between the source region 22a and the drain region 22b is increased. Here, a degree of potential damping of biomolecules charged by an ionic shielding within the electrolyte solution is determined by Debye length. That is, the Debye length can be controlled to sense, by using the bio FET, whether or not various probe biomolecules 27 are immobilized and whether or not the probe biomolecules 27 are bonded with the target biomolecules with accuracy and reappearance. Accordingly, the present invention is characterized in that the Debye length is controlled on the gate 2 surface in the electrolyte solution to sense whether or not various probe biomolecules 27 are immobilized and whether or not the probe biomolecules 27 are bonded with the target biomolecules with accuracy and reappearance.

In a general FET, all surfaces excepting the gate 23 region are passivated to prevent an ionic diffusion within the electrolyte solution 20. The gate 23 region can be coated with Au 26 to immobilize the probe DNAs 27, for example. A predetermined voltage is applied to the source region 22a and the drain region 22b of the bio FET positioned within the electrolyte solution 20. The charged biomolecules 27 are immobilized on an Au layer 26 of the gate 23 or are hybridized with the biomolecules (probes) 27 immobilized on the gate 23 surface, to directly affect the current flowing via the channel region between the source region 22a and the drain region 22b. At this time, the charged biomolecules immobilized or hybridized in the electrolyte solution differently effectively affect the channel region of the bio FET depending on their Debye lengths.

An ionic strength or a temperature of charged ions is varied in the electrolyte solution to control the Debye length. For example, if the ionic strength is increased, the Debye length can be decreased. If the ionic strength is decreased, the Debye length can be increased. Additionally, if the temperature is decreased, the Debye length can be decreased. Accordingly, in case where the probe biomolecules 27 are immobilized on the gate 23 surface, the temperature is decreased or the ionic strength is increased to reduce the Debye length, thereby accurately sensing an immobilization density and whether the immobilization of the probe biomolecules or not. Additionally, in case where the target biomolecules are hybridized with the probe biomolecules 27, the Debye length is relatively increased.

Hereinafter, a method of controlling the Debye length and sensing whether or not the biomolecules are bonded using the bio FET when the probe biomolecules 27 are immobilized on the gate 23 of the bio FET or when the target bio molecules are hybridized with the probe biomolecules 27 according to the present invention is described in detail.

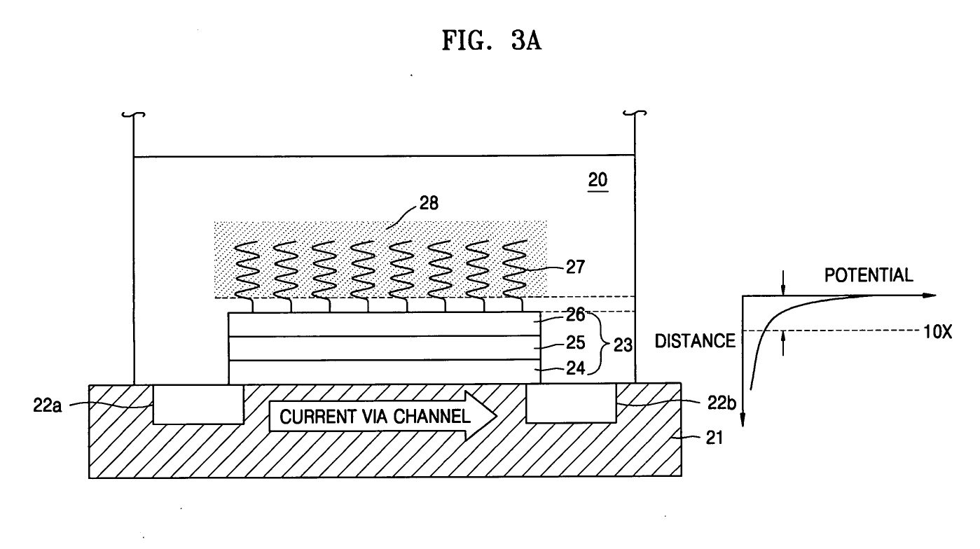

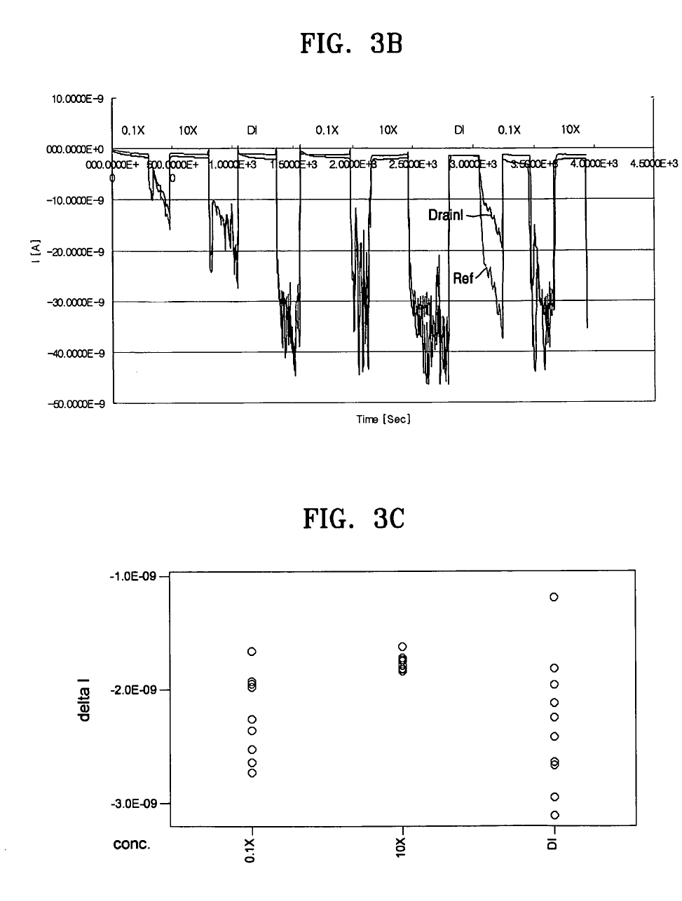

Referring to FIGS. 3A and 3B, the control of the Debye length in case where the probe biomolecules 27 are immobilized on the gate 23 surface is in detail described.

Here, the gate 23 is comprised of an oxidation layer 24, the poly silicon layer 25 and a metal layer (Au layer) 26. P-channel bio FET is used, and a reference bio FET has polymer coated on the gate electrode layer 25 to prevent the immobilization of the biomolecules. As the biomolecules, thiol-DNA(5′-thiol(C6)-GTTCTTCTCATCATC-3′, 30mer) is immobilized on the gate 23 surface, and an immobilization time is 60 minutes. As the electrolyte solution, Phosphate Buffered Saline (PBS) is used.

Here, it is sensed whether or not the probe DNA is immobilized on the gate 23 surface. In order to vary the Debye length, the ionic strength is varied. As such, after the Debye length on a substrate surface is controlled in the electrolyte solution, a current is sensed between the source region 22a and the drain 22b. This experiment confirms the effect of the Debye length on the biomolecules, and assures a sensing condition adaptive to the immobilization sensing. An experiment sequence for this is as follows.

a1. Thiol DNA is immobilized on the gate 23 surface for about 60 minutes in 0.1×PBS solution obtained by diluting a strength X of initial electrolyte solution. After it is confirmed from the current variation whether or not the thiol DNA is immobilized, a current (Ids) of a bio FET is sensed in 10×PBS solution for about 5 minutes.

a2. Next, the initial electrolyte solution is exchanged with the 10×PBS solution having ten times as much as the ionic strength X of the initial electrolyte solution, and the current flowing via the channel region of the bio FET in the 10×PBS solution is sensed for 5 minutes.

a3. After that, the solution is exchanged with deionized water (DI), and the current flowing via the channel region of the bio FET is sensed for 5 minutes.

a4. Continuously, the current is sensed in a sequence of the 0.1×PBS solution, the 10×PBS solution and the DI, to confirm the reappearance.

Here, a component of the 10×PBS solution is analyzed as shown in Table 1. The 0.1×PBS solution is obtained by diluting at 100 times as much as the 10×PBS solution. For reference, the 10×PBS solution is at pH 7.4±0.05.

TABLE 1

| Components | Strength (mg/L) | Molarity (Mm) |
|---|---|---|
| KH₂PO₄ | 1440.0 | 10.6 |
| NaCl | 90000.0 | 1551.7 |
| Na₂HPO₄ | 7950.0 | 29.6 |

A result of the above experiment is shown in FIG. 3B and Table 2. Table 2 a result data of the same experiment for two bio FETs.

TABLE 2

| | Chip 1 | | | Chip 2 | | |
|---|---|---|---|---|---|---|
| Trial | 0.1X PBS | 10X PBS | 0X DI | 0.1X PBS | 10X PBS | 0X DI |
| 1 | −1.82E − 09 | −1.84E − 09 | −2.18E − 09 | −1.66E − 09 | −1.63E − 09 | −1.82E − 09 |
| 2 | −2.05E − 09 | −1.92E − 09 | −3.39E − 09 | −1.93E − 09 | −1.72E − 09 | −1.20E − 09 |
| 3 | −4.04E − 09 | −1.79E − 09 | −2.42E − 09 | −2.52E − 09 | −1.82E − 09 | −2.64E − 09 |
| 4 | −2.32E − 09 | −1.78E − 09 | −2.98E − 09 | −1.95E − 09 | −1.79E − 09 | −2.67E − 09 |
| 5 | −2.78E − 09 | −1.83E − 09 | −2.75E − 09 | −1.93E − 09 | −1.75E − 09 | −3.11E − 09 |
| 6 | −2.95E − 09 | −1.88E − 09 | −3.21E − 09 | −2.36E − 09 | −1.74E − 09 | −2.95E − 09 |
| 7 | −3.03E − 09 | −1.79E − 09 | −2.65E − 09 | −2.26E − 09 | −1.79E − 09 | −2.12E − 09 |
| 8 | −2.54E − 09 | −1.75E − 09 | −3.32E − 09 | −2.73E − 09 | −1.84E − 09 | −2.42E − 09 |
| 9 | −2.02E − 09 | −1.84E − 09 | −2.77E − 09 | −2.64E − 09 | −1.82E − 09 | −1.96E − 09 |
| 10 | −2.53E − 09 | −1.76E − 09 | −2.15E − 09 | −1.98E − 09 | −1.84E − 09 | −2.25E − 09 |
| Mean | −2.61E − 09 | −1.82E − 09 | −2.78E − 09 | −2.20E − 09 | −1.78E − 09 | −2.32E − 09 |
| Stdev | 6.465E − 10 | 5.396E − 11 | 4.458E − 10 | 3.591E − 10 | 6.677E − 11 | 5.729E − 10 |
| CV[%] | 2.48E + 01 | 2.97E + 00 | 1.60E + 01 | 1.63E + 01 | 3.76E + 00 | 2.47E + 01 |

Table 2 shows a value of the current between the source region 22a and the drain region 22b, and a standard of deviation (Stdev) and a coefficient of variance (CV). Referring to Table 2, the same experiment is performed ten times using each of two chips to sense the current flowing via the channel region between the source region 22a and the drain region 22b. As a result, in a condition of the 10×PBS solution, a constant value can be obtained and a lowest CV can be obtained. In this result, in the 0.1×PBS solution, the Debye length on the gate 23 surface is increased about ten times as much as the 10×PBS solution, and the current flowing via the channel region is highly varied, and the CV is also relatively large. Accordingly, it is advantageous in the reappearance and the accuracy that it is sensed whether the probe biomolecules 27 are immobilized with the Debye length being reduced. That is, by reducing the Debye length to sense only a potential variation on the gate 23 surface, a noise factor and the like caused by an external charge can be minimized to obtain a result value with greater accuracy and reappearance. Referring to FIG. 3B, by comparing the sensed current of the reference bio FET with the sensed current of the bio FET where the probe DNA 27 is immobilized, a result of the 10×PBS solution is reappeared. Additionally, referring to FIG. 3C, in case where the probe DNA 27 is immobilized on the gate 23 surface, the current in the 10×PBS solution is sensed with the reappearance in comparison with the current in the 0.1×PBS solution and the DI. This shows that a better result is provided when the ionic strength is increased to reduce the Debye length in case where the probe biomolecules are sensed as described above.

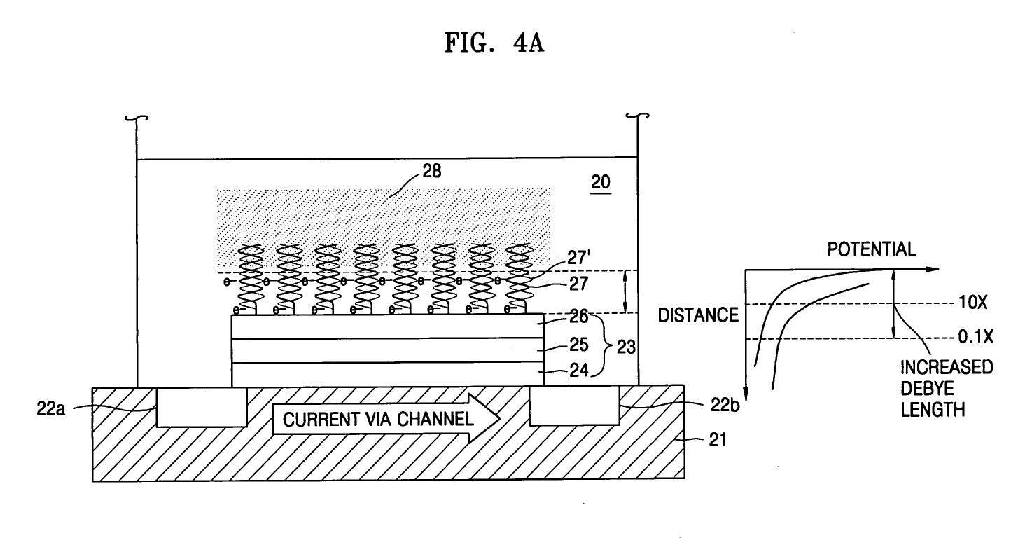

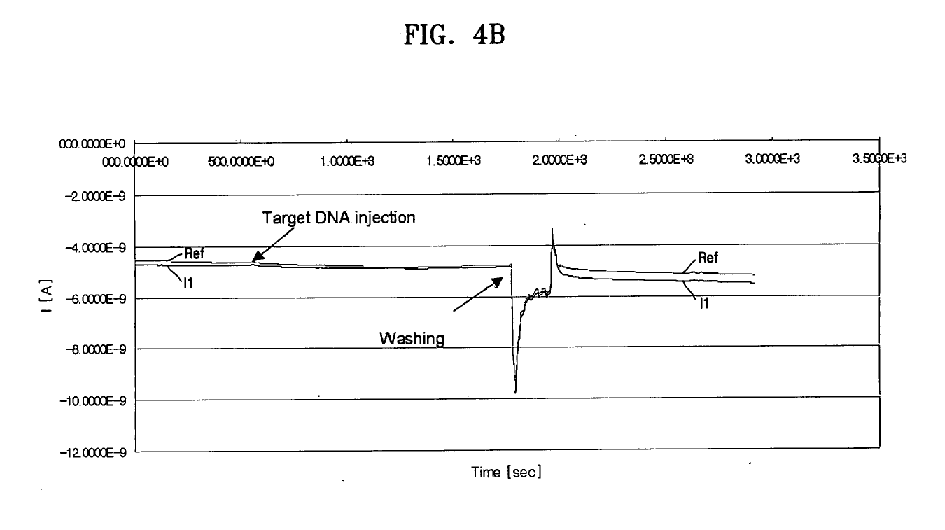

Next, an experiment is performed to sense whether or not the probe biomolecules are hybridized with the target biomolecules. This is illustrated in FIGS. 4A and 4B. At this time, the experimental condition is the same as those of immobilization experiments of the probe biomolecules 27 of FIGS. 3A through 3C, and Mercaptohexanol (MCH) is used as a spacer to enhance a hybridization efficiency of the probe biomolecules 27 and the target biomolecules 27′. At this time, an experiment sequence is as follows.

b1. Awaiting until the bio FET where the probe DNA is immobilized is stabilized in the 0.1×PBS solution.

b2. Injecting the target biomolecules, that is, the target DNA together with the 0.1×PBS solution to maintain to perform the hybridization with the probe DNA for about 2 hours.

b3. Deriving a hybridization reaction while injecting the 0.1×PBS solution, and sensing the current flowing via the channel region between the source region 22a and the drain region 22b through all processes of the experiment.

In this experiment, in case where it is sensed whether or not the probe biomolecules 27 immobilized on the gate 23 surface are hybridized with the target biomolecules 28, the result is shown with the accuracy and reappearance. When it is sensed whether or not there is the hybridization, the Debye length is increased in comparison with the immobilization of the probe biomolecules to accurately sense the effect of charges added by the target biomolecules bonded due to the hybridization.

FIG. 4B illustrates the variation of the current between the source region 22a and the drain region 22b sensed in real time when the hybridization is performed in the 0.1×PBS solution. After washing is performed and a predetermined time is lapsed, the current flowing between the source region 22a and the drain region 22b is increased by about 0.32 nA since an amount of surface charges on the gate 23 surface is increased by the hybridization of the target DNA and the probe DNA. However, the result cannot be obtained with the reappearance as a result of performing the above experiment in the 10×PBS solution. This is because it is difficult to sense whether or not the hybridization is performed in case where the Debye length is decreased by applying a high ionic strength of solution. This difficulty is because a distance representing an increase of the charges caused by the target DNA 27′ is increased from the gate 23 surface by hybridizing the probe DNA 27 with the target DNA 27′. Accordingly, in case where the bio FET is used to immobilize the probe biomolecules 27 on the gate 23 surface, the Debye length may be reduced. In case where the probe biomolecules 27 are hybridized with the target biomolecules 27′, the Debye length may be increased.

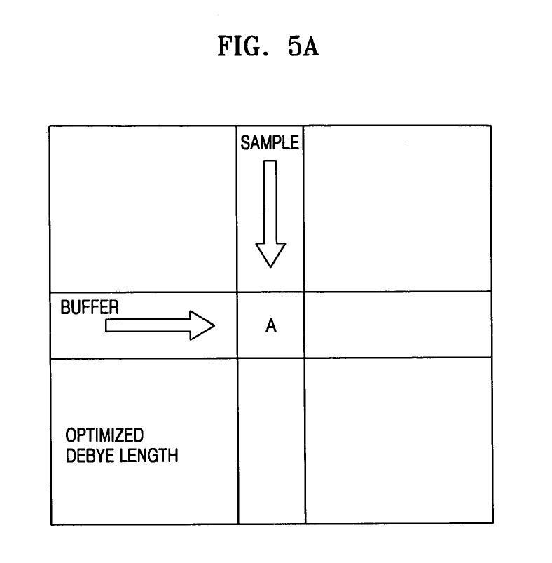

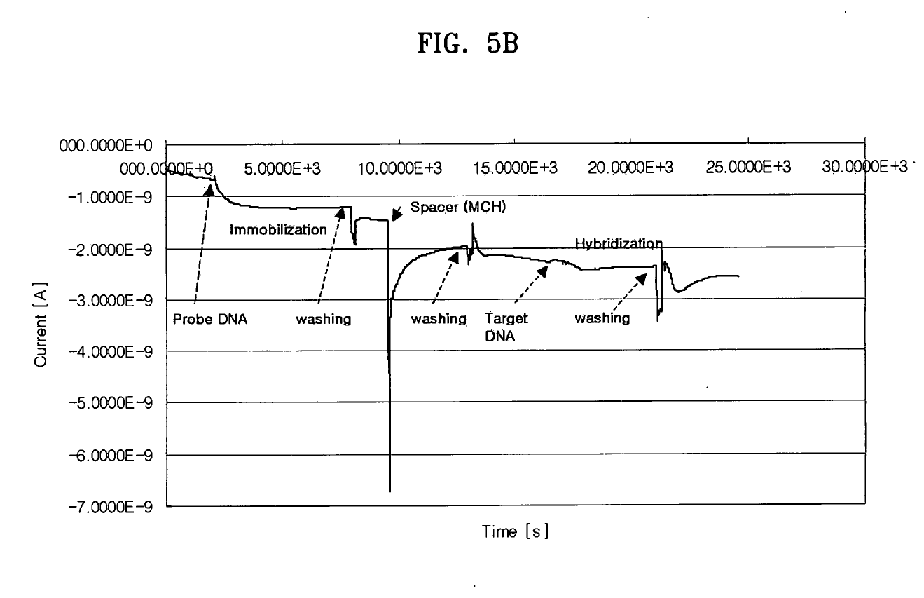

FIGS. 5A and 5B illustrates a method of sensing whether or not the biomolecules are immobilized to and hybridized with the bio FET in an optimal condition obtained through the experiment result of the immobilization and the hybridization of the biomolecules.

The above experiment condition is the same as that of FIGS. 3A through 3C. In order to increase an efficiency of hybridization, the MCH is used as the spacer. In order to inject and discharge the electrolyte solution and the washing solution to and from the gate 23 surface, the bio FET having a channel structure with an inlet port and an outlet port provided on the gate surface as shown in FIG. 5A is used. The experiment sequence is in detail described as follows.

c1. Awaiting until a signal of a bio FET sensor is stabilized, and then sensing the current flowing via the channel region between the source region 22a and the drain region 22b.

c2. Immobilization of the Probe DNA

The probe DNA is injected through a micro channel to the gate surface together with the 10×PBX solution. Additionally, the probe DNA is immobilized to the gate surface for about 60 minutes.

c3. The 10×PBS solution is injected through the micro channel for washing.

c4. Awaiting until a signal of a bio FET sensor is stabilized, and then sensing the current flowing via the channel region between the source region 22a and the drain region 22b is.

c5. Injecting the MCH, which is the spacer, into the gate surface through the micro channel together with the 0.1×PBS solution, and then awaiting for about 60 minutes.

c6. The 0.1×PBS solution is injected through the micro channel for washing.

c7. Awaiting until a signal of a bio FET sensor is stabilized, and then sensing the current flowing via the channel region between the source region 22a and the drain region 22b.

c8. Hybridization of the Probe DNA and the Target DNA

The target DNA is injected to the gate surface through the micro channel together with the 0.1×PBS solution. The probe DNA and the target DNA are hybridized for about 2 hours.

c9. The 0.1×PBS solution is injected through the micro channel for washing.

c10. Awaiting until a signal of a bio FET sensor is stabilized, and then sensing the current flowing via the channel region between the source region 22a and the drain region 22b.

An experiment of immobilizing and hybridizing the probe DNA to and with the target DNA through the above method for three bio FET chips is performed to sense the current. The experiment result is shown in Table 3. At this time, each of the chips has the bio FET and the reference bio FET having the polymer coated on the gate surface.

TABLE 3

| | I (immobilization)[A] | I (hybridization)[A] |
|---|---|---|
| Chip 1 − 1 | 3.23E − 10 | 2.21E − 10 |
| Chip 1 − 2 | 2.98E − 10 | 3.25E − 10 |
| Chip 2 − 1 | 1.51E − 10 | 2.15E − 10 |
| Chip 2 − 2 | 1.91E − 10 | 1.53E − 10 |
| Chip 3 − 1 | 2.13E − 10 | 2.45E − 10 |
| Chip 3 − 2 | 1.43E − 10 | 2.36E − 10 |

Here, current values sensed in the operations of c1, c4, c7 and c10 for each of the chips (chip 1, chip 2 and chip 3) are shown. The Debye length is controlled through the above process to represent the optimal condition when the immobilization and the hybridization of the biomolecules are sensed.

As described above, the present invention provides an effect in that the bio FET is used in the electrolyte solution to sense, with the enhancement of reappearance and accuracy, whether or not the biomolecules are immobilized to the gate surface and whether or not the probe biomolecules are hybridized with the target biomolecules. In a Lab on Chip, it is required to sense various biomolecules. At this time, the Debye length is varied depending on a sensed material and phenomenon to provide the optimal sensing condition, thereby providing the result with an excellent accuracy and reappearance. Further, the Debye length can be varied in real time to continuously sense other materials and phenomena by one bio FET.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of sensing biomolecules in an electrolyte solution by using a bio FET, wherein when it is sensed that probe biomolecules are immobilized to a gate surface of the bio FET or it is sensed that the probe biomolecules are hybridized with target biomolecules, a Debye length from the biomolecules having charges attached to the gate surface is controlled.

2. The method of claim 1, wherein when it is sensed that the probe biomolecules are immobilized to the gate surface of the bio FET, the Debye length is decreased.

3. The method of claim 1, wherein when it is sensed that the immobilized probe biomolecules are hybridized with the target biomolecules, the Debye length is increased.

4. The method of claims 1, wherein the Debye length is varied by controlling an ionic strength of the electrolyte solution.

5. The method of claim 1, wherein the Debye length is varied by controlling a temperature.

6. A method of sensing biomolecules in an electrolyte solution by using a bio FET, the method comprising:
when it is sensed that probe biomolecules are immobilized to a gate surface, adding a high ionic strength of solution to the electrolyte solution to reduce a Debye length from the probe biomolecules having charges, and then sensing a current flowing via a channel region between a source region and a drain region of the bio FET; and
when it is sensed that target biomolecules are hybridized with the probe bio molecules, adding a low ionic strength of solution to the electrolyte solution to increase the Debye length from the target biomolecules having charges, and then sensing a current flowing via the channel region between the source region and the drain region of the bio FET.
